(19) United States
(12) Patent Application Publication
(10) Pub. No.: US 2006/0134201 A1
(43) Pub. Date: Jun. 22, 2006

(54) Collagen troches for treating mouth lesions

(76) Inventor: Jeffrey T. Haley, Mercer Island, WA (US)

Correspondence Address: GRAYBEAL, JACKSON, HALEY LLP, 155 - 108TH AVENUE NE, SUITE 350, BELLEVUE, WA, 98004-5901, US

(21) Appl. No.: 11/016,474

(22) Filed: Dec. 16, 2004

(51) Int. Cl. A61K 38/38; A61K 9/20

(52) U.S. Cl. 424/464; 514/12

(57) ABSTRACT

A soluble/erodable troche for delivering collagen in the mouth for treatment of mouth lesions. The collagen may be gelatin from animal skins. The structure of the troche may be formed with a porous network that remains solid at human mouth temperatures and slowly dissolves/erodes in saliva. In some embodiments, the network is elasto-plastic and in some cases it is hydrophilic. The collagen is located within pores of the network. The troche may be formed by mixing and hydrating the ingredients, bringing them to above an activation temperature below boiling, and cooling them to form a gel. The hot mixture may be poured into molds, including a flat sheet, and the mold may also serve as packaging for delivery. The packaging may comprise a germ barrier moisture passing film which allows the troche to dry out or become re-hydrated without growing mold.

This application claims priority from PCT/US03/34549 filed Oct. 29, 2003, and from U.S. Ser. No. 10/287,843 filed Nov. 5, 2002 which is a CIP of Ser. No. 10/236,289 filed Sep. 4, 2002, and claims priority from U.S. 60/344,577 filed Dec. 28, 2001.

### BACKGROUND

For treatment of health problems in the mouth or throat, people have for centuries held in their mouths a composition containing herbal medication or other medication for topical application. The oldest name for such a composition, derived from Latin and previously from Greek, is “troche”. A modern form of troche is the cough drop, so named because it was formed by “dropping” hot, viscous, sugar-based candy onto a sheet or into a mold where it cools to form the troche. Another modern form of troche is the throat “lozenge”, so named because it was in the shape of a diamond (like on playing cards), which is the meaning of the word “lozenge”. The structural characteristics of these types of troches are determined by their primary structural ingredients which are typically corn syrup or sugars, including sugar alcohols. These troches are only mildly adherent to teeth and not significantly adherent to gums, cheeks, or lips.

To achieve higher concentrations of medication at a particular spot in the mouth than traditional troches can deliver, adherent troches in the form of adherent oral patches have been developed. An oral patch typically includes one or more flexible layers that do not dissolve entirely such as invented by Anthony et al. and disclosed in U.S. Pat. No. 5,713,852. Another example of an oral patch is the DentiPatch which has one or more non-soluble thermo-plastic layers and lidocaine, offered for sale by Noven Pharmaceuticals, Inc. As used herein, the word “patch” does not include preparations that remain free to move about the mouth rather than adhering in one place, such as cough drops or throat lozenges. Nor does it include preparations that do not hold together as a single item when held in the mouth such as preparations of powder, liquid, paste, viscous liquid gel, or a tablet or troche that crumbles into a powder or paste when chewed or placed in saliva.

The most significant differences between an oral patch as used herein and other forms of oral medicinal topical preparations are that an oral patch is designed to (1) release medication into the mouth over a relatively long period of time, such as 30 minutes or more, (2) be at least mildly adherent so that it can be placed in a preferred location and not be dislodged by gravity or gentle movement, and (3) remain in the mouth as a single item that will not spread to be in a plurality of locations in the mouth at one time.

There are many uses for preparations containing a medication to be delivered topically in the mouth. In many treatment situations, it is advantageous to retain the preparation at one location in the mouth rather than allowing it to move in the mouth such as when talking. U.S. Pat. No. 6,139,861 issued to Mark Friedman surveys the known methods for adhering a slowly dissolving medication to a location within the mouth. These methods include two forms of adherent soluble patches, referred to by Friedman as “mucoadhesive erodible tablets”. These tablets are formed using polymers carboxymethylcellulose, hydroxymethylcellulose, polyacrylic acid, and carbopol-934.

### SUMMARY OR THE INVENTION

In one aspect, the invention is a troche (which may also be a patch) that, when held in a human mouth, remains in the mouth as a single item that will not spread to be in a plurality of locations in the mouth at one time, lasts for more than 30 minutes, and releases collagen over time, with or without also releasing a medication. Collagen molecules, such as in the form of food grade gelatin, adhere very well to themselves and therefore adhere very well to the tissues of the mouth lining which are also made of collagen molecules. As the collagen molecules erode off the troche, they form a coating on nearby oral surfaces. If there is a lesion, such as an ulcer, this coating is soothing to the lesion. This troche requires use of binder materials that have suitable dissolution (erosion) characteristics. By selecting suitable binders and ratios of the ingredients, the troche will continually release collagen while it erodes. The binder ingredient may be a combination of gums that dissolve or erode in saliva, such as gum Arabic, carrageenan, xanthan gum, konjac gum, agar, or locust bean gum and non-dissolving food fibers such as cellulose.

The troche may be made of two primary components.

The first component is a porous molecular network formed as a unitary solid structure that remains a solid at human mouth temperatures, in contrast to being crumbly or a paste. The network is preferably hydrophilic so that, even when applied to a wet mucosal surface in the mouth, it will tend to adhere by absorbing moisture from the mucosal surface. Preferably, the network slowly dissolves or erodes in saliva so that the patch dissipates over time and the patch never has to be removed from the mouth.

The network component may be comprised of a thermo gel having a melting temperature higher than human mouth temperatures. Preferably, the thermo gel is elasto-plastic, such as formed by a mixture of the hydrogels konjac gum and xanthan gum dissolved in hot water and then cooled to form an elasto-plastic gel. Alternatively, the network may be comprised of a complex carbohydrate, such as cellulose, pectin, maltodextrin, or starch from potato, rice, corn, or wheat. Also, the network may be comprised of a hydrogel with a melting temperature higher than temperatures in the human mouth formed of amino acids, such as peptides.

The second component is the collagen which is distributed throughout the pores of the network. Because the collagen is hydrophilic and liquid at human mouth temperatures, it will adhere very well to wet surfaces inside the mouth and is quite soft which provides a soothing feeling to any sensitive tissue such as canker sores. The collagen is preferably partially hydrolyzed such as gelatin from animal protein.

In embodiments wherein the troche is adherent and therefore is also an oral patch, the patch will adhere to teeth, gums, cheek, lips, or tongue without the user first drying saliva from the tissue. If the user merely places the oral patch in his or her mouth and presses it in the desired location for 10 to 40 seconds, it will adhere to the tissues that it has been touching without movement, even though those tissues are wet. If the user wants to use an oral patch in the lip or under the tongue, the oral patch can easily be removed for talking and then easily be replaced without using a towel or a mirror.

The collagen gels to a solid at room temperatures. This allows the oral patch to be removed from the mouth and placed on a smooth surface, such as thermoplastic film where it then gels. Once the collagen gels, the oral patch again becomes handleable with the fingers to return it to the mouth without being too sticky to handle or leaving a residue on the fingers or on the plastic film.

In another aspect, the invention is a method for manufacturing a troche that releases collagen over time. In this method, ingredients for forming the porous network, molecules of the collagen (and molecules of a medication, if desired) are mixed together with water. The mixture is heated to dissolve all ingredients, either before the ingredients are added together or after they are added together, and the mixture is then cooled, thereby causing the ingredients for forming a network to form the porous network as a unitary solid structure having the collagen and any medication within its pores. Before it is cooled, the hot mixture may be deposited into a mold of a suitable shape (which may be a flat sheet) to form the preferred unitary solid structure. The mold may be formed in powdered starch, as is well known in the candy making industry for forming gumdrops. Alternatively, the mold may be formed of a rigid material such as metal or plastic. If the mold is thin plastic or aluminum, it may also serve as packaging for delivery of the troche to the consumer.

### BRIEF DESCRIPTION OF THE DRAWINGS

FIG. 1a shows a side view of a soft, adherent, soluble troche, which may also be an oral patch.

FIG. 1b shows a top view of the same troche.

FIG. 2 shows, in representational form, the structure of the solid, porous network, including the pores.

### DETAILED DESCRIPTION

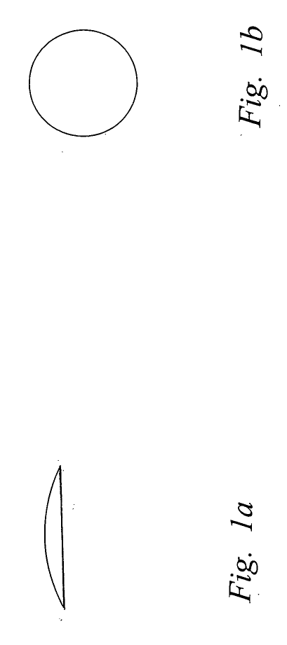

FIG. 1 shows a preferred shape for an embodiment of the troche, which may also be an oral patch. It is made with slowly dissolving (eroding) hydrocolloids so that that it typically lasts in the mouth for at least one to six hours. The troche can be formed in the shape of a tablet or a lozenge or a wafer or any other desired shape. A thin lentil shape is shown in FIG. 1a.

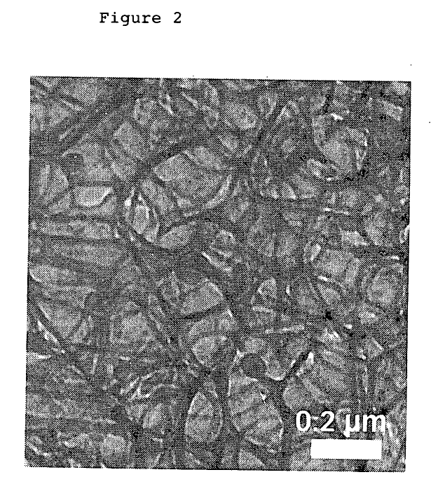

FIG. 2 shows, in representational form, the structure of the solid, porous network, including the pores. In one embodiment, a requirement for the network is that it remains a solid, rather than melting, at human mouth temperatures. So that the troche will slowly erode, the network should be made of a material with a low to moderate rate of disintegration in warm saliva. If the network does not erode fast enough, collagen will not be released fast enough to maintain the desired coating on nearby tissues, as the coating is constantly being washed away by saliva and tissue movement.

To understand by analogy how the porous network filled with collagen that is liquid at mouth temperatures becomes very sticky without disintegrating, imagine a fish net bag filled with linguini Alfredo. When the linguini Alfredo is cold, such as when just removed from a refrigerator, the Alfredo sauce is congealed and the entire structure is not very sticky. Imagine it is heated in a microwave oven. The Alfredo sauce melts and becomes quite sticky. By itself, the fishnet bag is not sticky. But, the holes are large enough that strands of linguini covered with sauce will bulge out of the holes. When warm, the entire structure, if thrown against a wall, would probably stick, yet the bag keeps it all together as one piece. The strands of linguini are like the long molecules of a polymer that is liquid at mouth temperatures. Their length keeps them from easily falling out of the fish net bag.

Many different compositions can be used to form the network. For ease of manufacturing, it is convenient if the network is comprised of a thermo gel having a melting temperature higher than human mouth temperatures. This allows the entire mixture to be a liquid at temperatures far above human mouth temperatures and allows the network to be formed by cooling the mixture such that the thermo gel forms the desired network by a gelation process. The temperature at which the gel forms can be lower than human mouth temperatures, provided the temperature at which it melts again is higher than human mouth temperatures.

Readily available materials that form such a gel include agar, in various forms, carrageenan, in most of its forms, particularly kappa carrageenan, konjac gum, locust bean gum, and xanthan gum. All of these materials form a thermo gel that is sufficiently elastic or plastic or a combination thereof for the network to feel soft in the human mouth if it is adequately hydrated. If water is dried out of the network, it will become hard and will produce an unattractive feel when placed in contact with sensitive tissues, such as canker sores. To prevent the network from drying out, it may be packaged with a hermetic seal or a non-evaporating plasticizer, such as glycerol (glycerin) may be added. However, the more glycerol is added the less adherent the oral patch will be.

Synthetic hydrogels may be used for the network. Protein-based hydrogels are usually prepared using proteins extracted from natural sources, but they may be synthesized, such as with diblock copolypeptide amphiphiles, as taught by Nowak, et. al, “Rapidly Recovering Hydrogel Scaffolds From Self-Assembling Diblock Copolypeptide Amphiphiles”. Nowak, A. P.; Breedveld, V.; Pakstis, L.; Ozbas, B.; Pine, D. J.; Pochan, D.; Deming, T. J. Nature, 2002, 417, 424-428. The use of synthetic materials allows adjustment of copolymer chain length and composition. Synthetic hydrogels may also be made from polysaccharides and synthetic block copolymers which form thermoreversible gels and allow the solubilisation of hydrophobic medications for controlled release, as taught by Williams, Pa., at the Centre for Water Soluble Polymers, North East Wales Institute, Plas Coch, Mold Road, Wrexham, Wales.

Instead of forming the network with a true hydrogel, the network may be formed with a complex carbohydrate, such as cellulose, pectin, starch, maltodextrin or other polysaccharides. Forming of hydrated network structures out of such materials is well known in the candy making industry for making gummy candies. Or the network may be formed with a combination of a true hydrogel and a complex carbohydrate.

As the collagen molecules slough off the troche while it slowly dissolves (erodes), they tend to adhere to the nearby mouth lining, forming a coating. This coating significantly reduces the sensitivity of an ulcer or other lesion, both to touch and to chemical irritants. The collagen molecules may be partially hydrolyzed, making them shorter and lower in molecular weight, in the form of commercially available food grade gelatin which is made from animal skins.

Hydrated hydrolyzed collagen assists with wound healing in human skin. The inventor has found that, when applied in the mouth via an oral patch, it reduces pain and assists with healing of wounds and other lesions in the mouth. Oral patches made according to this invention with no active ingredient other than collagen were tested for short term pain reduction in aphthous ulcers with success equal to commercially available topical treatments having no anesthetic. Tests for speeding of healing of oral wounds showed small but significant improvement.

Commercially available gelatins are graded according to “bloom strength” which refers to the strength of the gel that is formed. Gelatin with a higher bloom strength (made with longer collagen molecules) is preferred for the adherent oral patch because it also has a higher viscosity in liquid form. The high viscosity in liquid form prevents the gelatin molecules from escaping the network substantially faster than the network erodes, makes a coating on mouth tissues that washes away more slowly, and better retains any medication for slow release. The highest commercially available bloom strength, 250, is preferred.

A preferred patch formulation is made by combining collagen and with binder ingredients that form a network. If the collagen is bound too tightly by binder ingredients, the patch will not be adherent enough to stay in place. If there is too much collagen, the patch will be too adherent for comfort, especially since the patch is adherent on both sides. If the entire compound is bound too tightly, it will not erode at a fast enough rate to release the active ingredients on a desired schedule. If the compound is not bound tightly enough, the patch will fall into pieces.

If the network is formed of a hydrogel as described above, the troches/oral patches may be manufactured by processes well known in the candy making industry. The process is to form a well-hydrated mixture at temperatures above the activation temperature and below the boiling temperature of water so that water does not boil off and yet the hydrogels are fully activated for gelling when the product is cooled. In this process, the network can be formed of a combination of a true hydrogel such as xanthan gum with locust bean gum or with konjac gum and a complex carbohydrate such as cellulose or pectin or starch. An effective range of ratios by dry weight is 30-80% gelatin, 5-30% cellulose fibers, and 2-15% gums such as kappa carrageenan or xanthan gum plus locust bean gum or konjac gum heated to between 130 and 200 degrees F. Having at least 50% collagen is preferred to provide large amounts of collagen to the mouth while limiting the size of the troche, but the collagen can not exceed 80% or the structure will not have enough strength to retain its shape and spread out the release of collagen over at least one hour. The water may be up to 80% of the wet mixture, increasing the amount of subsequent drying required.

Where a bulky ingredient is added and the troche should be sticky to be also a patch, it may be preferable to have as much of the ingredient as possible. But if there is too much bulk, the collagen will not be adherent enough or the binder will not be strong enough or both. This problem is not present if a concentrated added ingredient is used. So, for a bulky added ingredient, it is best to have very few other ingredients besides the ingredient, collagen, and binders. Also, many possible ingredients that might be added will reduce the adherence of the collagen, for example, oil, such as mineral oil or peppermint oil or menthol, and glycerin, even in very small quantities, will reduce adhesion.

Where the added ingredient is licorice root extract, testing shows that, if the binders are xanthan gum, konjac gum, and cellulose fiber, effective dry weight formulations have between 13% and 37% licorice extract, between 33% and 55% food grade gelatin, between 22% and 54% binders, and essentially nothing else but perhaps small amounts of concentrated flavoring.

The hot mixture is poured or squirted into molds. The molds be may open top molds, including a flat sheet, or closed molds. Open top molds may be formed by pressing a plug into powdered starch such as cornstarch or may be formed in a tray for packaging the products such as thermo formed PVC or PET or a cold press laminate of aluminum and PVC with a thin layer of polyamide for strength. Closed molds may be used such as in an injection-molding machine. The molds may be plastic lined, in which case the plastic becomes a part of the final packaging. A suitable size for each troche is 0.8 grams poured into the mold.

If the troches are deposited in powdered starch, the starch absorbs some of the excess water and the troches are further dried in a drying room before being removed from the starch and packaged in a hermetic seal. They may be sterilized with gamma radiation or heat and pressure in a retort.

If the troches are deposited in molds formed in a tray, the tray is stored in a drying room until the troches lose a suitable amount of moisture. A suitable method of drying in trays is to expose them with convection to room temperature and 30 to 50% humidity for 3-5 days. In the drying process, the troches lose most of their water, so an troche that started at 0.8 grams poured into the mold becomes 0.42 grams. The trays are then sealed with a film or foil lid that is adhered by conventional heat-sealing techniques. The entire package may be sterilized with gamma radiation or heat and pressure in a retort.

For most applications, most users prefer that the troches be medium dry to dry. With this starting dryness, the troches are more adherent and have more integrity so they can be removed for talking or eating and then replaced. The only drawback to this dryness is that the troche becomes hard when it dries, giving the troche a less soothing feel. It is also less conforming and therefore does not stick well to hard surfaces such as gums and teeth. When the troche is placed in contact with delicate tissue, such as a large canker sore, most users prefer that the troche be moist and soft. To address these concerns, the troches may be packaged with a film that allows moisture to pass so moisture can easily be added to or removed from the troches without removing them from the packaging. If the packaging film is a barrier to germs, this allows the oral patches to remain sterile and not grow mold even when they are moist. Effective films are cellophane, polystyrene, poybutadiene, polyamide, Tyvek (matted polyethylene threads) and expanded films such as Goretex. Polyamide with a thickness of 0.7 mil to 1.0 mil is effective. Allowing such a package to sit for a day or two with a few drops of water on the package is sufficient to hydrate the troche inside. Conversely, allowing the package to sit on a shelf in a dry room for one to three days is sufficient to dry out the troche. While particular embodiments of the invention have been described above, the scope of the invention should not be limited by the above descriptions but rather limited only by the following claims.

1. A troche for delivering collagen to mouth lesions, comprising:
(a) a porous network formed as a unitary solid structure that does not melt at human mouth temperatures; and
(b) molecules of collagen located within pores of the network; where
(c) the troche is between 30% and 80% collagen by dry weight.

2. The troche of claim 1 wherein the collagen is partially hydrolyzed.

3. The troche of claim 1 wherein the collagen is gelatin from animal protein.

4. The troche of claim 1 wherein the network is hydrophilic.

5. The troche of claim 1 wherein the network is comprised of a thermogel having a melting temperature higher than human mouth temperatures.

6. The troche of claim 1 wherein the network is comprised of at least one hydrogel selected from the group of konjac gum, xanthan gum, locust bean gum, agar and carrageenan.

7. The troche of claim 1 wherein the network is comprised of complex carbohydrate.

8. The troche of claim 5 wherein the thermogel is a mixture of konjac gum and xanthan gum dissolved in hot water and then cooled to form an elasto-plastic gel.

9. The troche of claim 1 wherein the network is comprised of cellulose.

10. The troche of claim 1 wherein the network is comprised of amino acids.

11. A troche for delivering collagen to mouth lesions, consisting essentially of:
(a) 30-80% collagen,
(b) 5-50% food binders, and
(c) 0-50% other ingredients.

12. The troche of claim 11 wherein the collagen is partially hydrolyzed.

13. The troche of claim 11 wherein the collagen is gelatin from animal protein.

14. The troche of claim 11 wherein the food binders comprise at least one of food fibers and food gums.

15. The troche of claim 14 wherein the food gums are comprised of at least one hydrogel selected from the group of konjac gum, xanthan gum, locust bean gum, agar and carrageenan.

16. The troche of claim 11 wherein the food binders are comprised of complex carbohydrate.

17. The troche of claim 14 wherein the food fibers are comprised of cellulose fibers.

18. A method for manufacturing a troche for delivering collagen in a human mouth over time, comprising:
(a) mixing a mixture comprising ingredients for forming a hydrophilic porous network with a rate of disintegration in saliva that remains a solid at human mouth temperatures, collagen molecules, and water;
(b) heating the mixture; and
(c) cooling the mixture, thereby causing the ingredients for forming a network to form a hydrophilic porous network as a unitary solid structure having the collagen within its pores.

19. The method of claim 18 wherein the network is formed by a process of gelation resulting from the cooling step.

20. The method of claim 18 wherein the collagen is gelatin rendered from animal tissue.

21. The method of claim 18 further comprising, between the heating step and the cooling step, depositing hot mixture into a mold and the cooling step is performed with the mixture in the mold.

22. The method of claim 21 wherein the mold is formed in powdered starch.

23. The method of claim 21 wherein the mold is a flat, rigid sheet of plastic film.

24. The method of claim 19 further comprising packaging the troche in surrounding material that comprises a germ barrier moisture-passing film.
